(19) United States
(12) Patent Application Publication
(10) Pub. No.: US 2026/0245382 A1
(43) Pub. Date: Aug. 20, 2026

(54) SYSTEMS AND METHODS FOR MAGNETIC RESONANCE IMAGING CALIBRATION

(71) Applicants: The General Hospital Corporation, Boston, MA (US); PRESIDENT AND FELLOWS OF HARVARD COLLEGE, Cambridge, MA (US)

(72) Inventors: Yiqiao Song, Newton, MA (US); Xuanhui Sharron Lin, Newton, MA (US); Bruce R. Rosen, Lexington, MA (US)

(73) Assignees: The General Hospital Corporation, Boston, MA (US); PRESIDENT AND FELLOWS OF HARVARD COLLEGE, Cambridge, MA (US)

(21) Appl. No.: 19/473,101

(22) PCT Filed: Apr. 5, 2024
(86) PCT No.: PCT/US2024/023458

§ 371 Date: Oct. 6, 2025

Related U.S. Application Data
(60) Provisional application No. 63/494,876, filed on Apr. 7, 2023.

(51) Int. Cl. G06V 20/69; A61B 5/055; G06T 7/00; G06V 10/25; G16H 30/20; G16H 30/40

(52) U.S. Cl. CPC G06V 20/695; A61B 5/055; G06T 7/0012; G06V 10/25; G16H 30/20; G16H 30/40; G06T 2207/10092; G06T 2207/30024

(57) ABSTRACT

A method can include determining a first association between a first magnetic resonance imaging (MRI) characterization and a first histological characterization obtained from a first reference sample of a patient. The method can include determining a second association between a second MRI characterization and a second histological characterization obtained from a second reference sample of the patient. The method can include obtaining a third MRI characterization of a region of interest (ROI) from an MRI scan of the patient. The method can include determining a third histological characterization for the ROI using the third MRI characterization and at least one of the first association or the second association.

### CROSS-REFERENCE TO RELATED PATENT APPLICATION

This application claims the benefit and priority of U.S. Provisional Patent Application No. 63/494,876, filed on Apr. 7, 2023, the entirety of which is incorporated by reference herein.

### GOVERNMENT RIGHTS

This invention was made with government support under EB026996 awarded by National Institutes of Health (NIH) and under DE-AR0001063 awarded by U.S. Department of Energy (DOE). The government has certain rights in this invention.

### TECHNICAL FIELD

The present disclosure relates to magnetic resonance imaging, and in particular MRI in medicine and disease diagnostics.

### BACKGROUND

Magnetic resonance imaging (MRI) can be used in all areas of medicine for diagnostics and staging of diseases and for monitoring the progression of the diseases and treatment.

### SUMMARY

At least one aspect of the present disclosure is directed to a method. The method can include determining a first association between a first MRI characterization and a first histological characterization obtained from a first reference sample of a patient. The method can include determining a second association between a second MRI characterization and a second histological characterization obtained from a second reference sample of the patient. The method can include obtaining a third MRI characterization of a region of interest (ROI) from an MRI scan of the patient. The method can include determining a third histological characterization for the ROI using the third MRI characterization and at least one of the first association or the second association.

Another aspect of the present disclosure is directed to a method. The method can include performing a first MRI scan of a patient while a biopsy needle is inserted into the patient. The method can include extracting a sample from the patient using the biopsy needle. The method can include tagging a first portion of the sample with a first MRI characterization obtained from the first MRI scan. The method can include tagging a second portion of the sample with a second MRI characterization obtained from the first MRI scan. The method can include tagging the first portion of the sample with a first histological characterization. The method can include tagging the second portion of the sample with a second histological characterization. The method can include performing a second MRI scan of the patient to obtain a third MRI characterization of a ROI. The method can include determining a third histological characterization for the ROI using at least one of: the first MRI characterization, the second MRI characterization, the third MRI characterization, the first histological characterization, or the second histological characterization.

The summary is illustrative only and is not intended to be in any way limiting. Other aspects, inventive features, and advantages of the devices and/or processes described herein, as defined solely by the claims, will become apparent in the detailed description set forth herein and taken in conjunction with the accompanying drawings.

### BRIEF DESCRIPTION OF THE DRAWINGS

The details of one or more implementations of the subject matter described in this specification are set forth in the accompanying drawings and the description below. Other features, aspects, and advantages of the subject matter will become apparent from the description, the drawings, and the claims.

FIG. 1 illustrates simulated T2 weighted MRI data, according to an embodiment.

FIG. 2 illustrates the colocation of tissue type information, according to an embodiment.

FIG. 3 illustrates a method for MRI calibration, according to an embodiment.

FIG. 4 illustrates a method for MRI calibration, according to an embodiment.

FIG. 5 illustrates a block diagram of an architecture for a computer system that can be employed to implement elements of the systems and methods described and illustrated herein.

Like reference numbers and designations in the various drawings indicate like elements.

### DETAILED DESCRIPTION

Following below are more detailed descriptions of various concepts related to, and implementations of, methods, apparatuses, and systems for MRI calibration. The various concepts introduced above and discussed in greater detail below may be implemented in any of a number of ways, as the described concepts are not limited to any particular manner of implementation. Examples of specific implementations and applications are provided primarily for illustrative purposes.

MRI signals and images can be obtained from MRI scanners. MRI scanners can include equipment (e.g., hardware) such as magnets to produce a static magnetic field, RF electronics to generate pulses of RF magnetic fields, and/or gradient electronics to produce pulses of DC magnetic fields and gradients. The operation of this hardware in concert to produce signals can originate from the hydrogen atoms in samples or patients. The signals can be included in an MRI scan. The signals and/or results from the MRI scan can then be then processed to produce images.

The magnetic resonance (MR) phenomenon can involve the application of magnetic fields to an object that impacts the magnetic moment (e.g., spin) of an atom in the object. The magnetic field can cause the spin of the atoms in the object to align along and oscillate (e.g., precess) about the axis of the applied magnetic field. The spin magnetization of the atoms can be measured. The return to equilibrium of this magnetization (e.g., relaxation) can be of interest. For example, longitudinal relaxation due to energy exchange between the spins of the atoms and the surrounding lattice (e.g., spin-lattice relaxation) can be denoted by a time T1 when the longitudinal magnetization has returned to a predetermined percentage (e.g., 63%) of its final value. Longitudinal relaxation can involve the component of the spin parallel or anti-parallel to the direction of the magnetic field. Transverse relaxation that results from spins getting out of phase can be denoted by time T2 when the transverse magnetization has lost a predetermined percentage (e.g., 63%) of its original value. The transverse relaxation can involve the components of the spins oriented orthogonal to the axis of the applied magnetic field. The T2 measurement can be performed using the spin-echo pulse sequence. The spin-echo pulse sequence can involve one 90-degree pulse followed by one 180-degree refocusing pulse. T2 can be measured using Carr-Purcell-Meiboom-Gill (CPMG) pulse sequence, which can utilize an initial 90-degree excitation pulse followed by a series of 180-degree (pi) pulses.

The signals (and thus the images) can be affected by various physical phenomena of the molecular dynamics of the hydrogen-carrying molecules, such as water and fat molecules. The phenomena can include, for example, spin-lattice relaxation, spin-spin relaxation, and molecular diffusion of the molecules. Spin-lattice relaxation can be characterized by a time constant, T1. Spin-spin relaxation can be characterized by a time constant, T2. The diffusion can be characterized by a diffusion constant, D. Different tissues and/or tissues at different stages of a disease can exhibit different values of T1, T2, and/or D. A tissue sample may be characterized by a distribution of T1, T2 and D, or two-dimensional distributions such as T1-T2 and D-T2 correlations. All of these can be MRI parameters (e.g., MRI properties) and they can be experimentally measured and used to characterize tissue properties. For example, in brain tumors such as glioma, the tumor tissue can often exhibit a significantly longer T1 and T2 than normal brain tissues. As a result, such enhanced T1 and T2 values can be used as a signature of the tumor to quantify the size of the tumor and the stage of the tumor progression. During treatment, MRI images can provide evidence for the shrinkage of the lesion and thus the effectiveness of the treatment regimen. Such monitoring can be useful for monitoring and fine-tuning the treatment plan.

However, an MRI scan and MRI image may not be sufficient to determine the accurate value of the MRI parameters such as T1, T2, and/or D. This can be due to the need for multiple images in order to accurately determine the T1, T2, and/or D parameters. Multiple scans may not be frequently prescribed. In some cases, only qualitative determination of these parameters is available. For example, the T2-weighted MRI image can show a bright area for the longer T2 tissue, which can indicate a possible tumor. However, the image may not provide an accurate determination of this T2 value at each voxel. Furthermore, such MRI parameters may vary significantly among different patients. Thus, it may be a challenge to use clinical MRI to accurately determine tissue types and disease stages.

The systems and methods of the present disclosure can include combining clinical MRI scans with MRI measurements on tissue samples. This can be used to achieve accurate determinations regarding different tissue types and/or disease stages. The tissue samples can be obtained through biopsy or resection surgery. The fresh samples can be measured on an MRI device and/or be subject to pathological examination/analysis. The method can link the information of pathology and the MRI parameters of tissue samples from the same patient to achieve an improved interpretation of the clinical MRI images. When MRI is needed for additional follow-up examinations for the same patient, the systems and methods of the present disclosure can provide the patient-specific MRI calibration to achieve personalized and more accurate diagnostics. This method workflow can be specific to each individual patient and can be referred to as personalized MRI. The systems and methods of the present disclosure can combine (1) MRI measurement of tissue samples using small NMR/MRI systems and (2) clinical MRI scans. The combination can be used to accurately identify tissue types and disease stages for a specific patient to achieve personalized MRI.

MRI properties of tissues (e.g., sometimes referred to as MRI characterization or MRI parameters) can include spin-lattice relaxation time (T1), spin-spin relaxation time (T2), and diffusion coefficient (D). Each property can be measured by specific pulse sequences and MRI experiments.

For the T1 measurement, different methods can be used to obtain T1. The methods can include the inversion-recovery (IR) method and the saturation-recovery (SR) method. For the IR method, the pulse sequence can be described as Equation 1:

$$RD - p 180 - WT - p 90 - ACQ \qquad ( 1 )$$

Here, the first time period RD can be long (e.g., several times T1 of the sample) for the system to recover to thermal equilibrium. The p180 pulse can invert the magnetization. The time WT can allow the magnetization (M) to relax according to Equation 2:

$$M ( WT ) = {M}_{0} \{ 1 - 2 exp [ - \frac{WT}{{T}_{1}} ] \} , \qquad ( 2 )$$

Here, M₀ is the equilibrium magnetization of the sample. The magnetization can be measured after the data acquisition (ACQ) after the p90-degree pulse. Several measurements of the signal for a series of values of WT can be obtained to determine the T1 of the sample.

Furthermore, imaging pulse sequences combining RF pulses and gradient pulses can be applied after the WT in Equation 1 to produce MRI images. In this case, Equation 2 can be applied to each voxel of the image. When several images with different WT are obtained, it is possible to obtain T1 for each and all voxels in order to obtain a spatial map of T1 of the sample.

In the SR method, the pulse sequence can be described as Equation 3:

$$WT - p 90 - ACQ \qquad ( 3 )$$

Here it can be assumed that the magnetization at the beginning of the WT period is zero due to the saturation in the previous experiment or by additional pulses to saturate. The magnetization can recover during the time WT according to Equation 4:

$$M ( WT ) = {M}_{0} \{ 1 - exp [ - \frac{WT}{{T}_{1}} ] \} , \qquad ( 4 )$$

Similar to the IR method, the imaging method and pulse sequences can be added to obtain an image of the sample.

For the measurement of T2, a spin-echo pulse sequence can be used, which can be described in Equation 5:

$$RD - p 90 - TE / 2 - p 180 - TE / 2 - ACQ \qquad ( 5 )$$

Here the time period TE can be defined as echo time. The time spacing between the p90 and p180 is half TE (e.g., TE/2). The magnetization can decay as a function of TE according to Equation 6:

$$M ( TE ) = {M}_{0} exp [ - \frac{TE}{{T}_{2}} ] , \qquad ( 6 )$$

where M₀ is the magnetization when TE/T2 approaches 0. Similar to the T1 measurement described above, when several measurements with different TE are obtained, the value of T2 of a sample can be obtained.

In addition to the spin-echo sequence, the CPMG sequence can be used with multiple p180 pulses to generate a train of echoes. The echo time for echo number n is n*TE. For example, for the first echo, n=1 and for the 10^{th} echo, n=10. As a result, with one experiment, several data points of the signal decay can be obtained and thus accelerate the measurement of T2.

The property of molecular diffusion can reflect the molecular composition as well as the physical and fluidic environment. For example, when a water molecule is in a viscous fluid, its diffusion coefficient (D) can decrease. When fluid is inside porous materials or tissues, water diffusion can be restricted due to the presence of solid materials or membranes. The diffusion coefficient can be lower than the value in the bulk fluid. As a result, the measurement of diffusion coefficient has been used to characterize porous materials and tissue microstructure.

Diffusion can be measured using a spin-echo sequence (e.g., using Equation 5) with additional field gradient pulses during the two time periods, first between the p90 and p180 and second between p180 and ACQ. The magnetization decay due to diffusion can be described by Equation 7:

$$M ( b ) = {M}_{0} exp [ - bD ] , \qquad ( 7 )$$

where D is the diffusion coefficient, and b is the diffusion weighting determined by the pulse sequence and in particular the field gradient pulses used. Similar to the T1 and T2 measurements, several signals with different b values can be obtained in order to determine D. Furthermore, these experiments can be used to produce distributions of T1, T2 and D, and these experiments can be combined to produce the two-dimensional correlation functions such as the T1-T2 correlation.

To obtain T1, T2, and/or D, several data and/or images with different WT, TE, or b values can be obtained. However, in clinical MRI applications, MRI can often be performed with a single WT, TE, or b value to reduce the total examination time or cost. Clinical MRI can include MRI for the observation and treatment of patients. Such MRI scans are often called T2-weighted (T2w-) MRI, T1-weighted (T1w-) MRI, or diffusion-weighted MRI (DWI). Even though such an imaging method may not allow for accurate determination of T1, T2, or D values, the method can still be useful.

This is because even though an accurate determination of T1, T2, and D is not available, the different tissues with different parameters can appear with different intensities on an image. In a T2-weighted image of a brain cancer patient, different parts of the anatomy can show different brightness reflecting the different signals. The middle region of the ventricles can be filled with cerebrospinal fluid (CSF). The T1, and T2 of CSF can be long (e.g., approximately 1-2 seconds) and D can be high (e.g., approximately 2-3*10-9 m²/s, similar to that in bulk water). The T2 of gray and white matter can be much shorter than that of the CSF. For example, the T2 can be approximately 0.060 seconds to 0.2 seconds. As a result, for a given TE (e.g., TE=109 ms), the decay in the CSF region can be much less than that in the other brain regions. As a result, the ventricles can appear very bright with high intensity and the gray and white matter regions can appear much darker. The tumor region can be brighter than the surrounding tissues, and as a result, the tumor region can be identified. The tumor regions may not be as bright as the ventricle, which can indicate that the T2 of the tumor is shorter than the T2 of CSF.

The appearance of an image can be highly dependent on the actual MRI sequence used. For example, using a FLAIR sequence where the long T1 signal is suppressed, the ventricle can appear dark (e.g., low signal) and the shorter T2 signal regions can appear bright (e.g., high signal). A radiologist can be trained to recognize the different behaviors for each MRI sequence. However, it may be difficult to identify disease tissues when the signal is diffusive and/or the boundary is continuous and not very clear. When two regions show similar brightness, it can be difficult to identify the disease tissue. As a result, a more quantitative analysis can be beneficial to help distinguish different tissues. For example, the analysis can distinguish between normal tissue and tumor tissue. The analysis can distinguish between benign tissue and cancerous tissue.

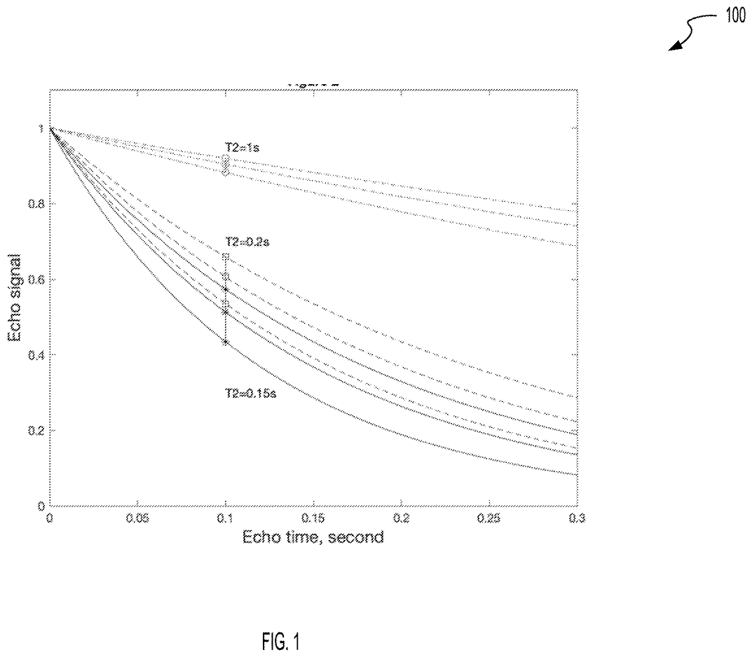

An example can be used to illustrate a quantitative analysis to identify tissues more accurately. Consider a T2-weighted MRI experiment and an echo time of 0.109 seconds. FIG. 1 shows the simulated data for three signals with T2 of 0.15 seconds, 0.2 seconds, and 1 second to represent three tissue types (e.g., type 1, 2, and 3, respectively). For the three types of tissues, an inaccuracy of 20% for T2, can be considered. This can be illustrated by the three lines for each tissue. The data points (e.g., dots and squares) can indicate the potential signal range for either tissue type for this specific MRI sequence. As a result, tissue 1 and tissue 2 can show significant overlap and it can be difficult to distinguish them from the above MRI experiment. Thus, more accurate tissue types can be obtained provided more accurate T2 values of the different tissue types are known. For example, if T2 has been determined to be 0.15 seconds and 0.2 seconds (center lines of the solid and dashed line groups), the tissue 1 and tissue 2 can be readily distinguished by the MRI experiment.

FIG. 1 illustrates simulated T2 weighted MRI data for T2 values of 0.15 seconds, 0.2 seconds, and 1 second for three types of tissues (type 1, 2, and 3 respectively). Type 1 tissue (e.g., tissue 1) is shown using solid lines. Type 2 tissue (e.g., tissue 2) is shown using dashed lines. Type 3 tissue (e.g., tissue 3) is shown using dot-dashed lines. For each tissue type, three different lines are shown to indicate the range (e.g., 20%) of possible T2 for each tissue type. Given such range of each tissue, signals from tissue 1 and tissue 2 may not be reliably identified, while tissue 3 is sufficiently different from tissue 1 and tissue 2. The symbols indicate the echo time for MRI, also indicating overlaps of signals among tissue 1 and tissue 2.

Achieving more accurate tissue identification can rely on the accurate assessment of the T1, T2, and/or diffusion properties and other MRI parameters of the various tissue types for each individual patient. Such determination of the T1, T2, and/or diffusion and other MRI parameters for individual patients can reduce the uncertainty of these parameters obtained from a population of patients. This process can be known as the calibration of MRI for individual patients.

Measurement (e.g., MRI characterization) of tissue samples using small NMR/MRI systems may include measurement of some or all MRI parameters, such as T1, T2, D, and their distributions and/or correlations. Accurate measurement of these MRI parameters can allow for the prediction of signals for the clinical MRI scan for each tissue and thus achieve high tissue resolution.

A biopsy can include a medical test performed by a surgeon, interventional radiologist, or interventional cardiologist. Biopsies can be performed to determine whether the tissue of a tumor is malignant or the cause of an unexplained infection or inflammation. The process can involve the extraction of sample cells or tissues for examination to determine the presence or extent of a disease. The tissue can be examined under a microscope by a pathologist. The tissue may be analyzed chemically. A variety of biopsy techniques can be applied for cancer diagnosis. When an entire lump or suspicious area is removed, the procedure is called an excisional biopsy. An incisional biopsy or core biopsy can sample a portion of the abnormal tissue without attempting to remove the entire lesion or tumor. When a sample of tissue or fluid is removed with a needle in such a way that cells are removed without preserving the histological architecture of the tissue cells, the procedure can be called a needle aspiration biopsy. A fine-needle aspiration biopsy can include the removal of tissue fluid or a very small piece from a tumor using a thin needle, and this type of biopsy can provide a diagnosis without surgical intervention. When the specimen is evaluated, in addition to diagnosis, the amount of uninvolved (e.g., normal) tissue around the lesion can be examined and compared.

Minimally invasive tissue sampling modalities can include fine-needle aspiration (FNA) and core needle biopsy (CNB). They can be in contrast to larger and more invasive surgical incisional/excisional biopsies. Although similar in many ways, there can be differences in the collection, processing, interpretation, and suitability for ancillary testing that exist between FNA and CNB.

The cellular material can be obtained through needle puncture, either relying on the forward motion and the intrinsic capillary action of an FNA versus the spring-loaded physical cutting action of a tissue core by CNB. The external diameter of the needle can be described by the respective needle gauge, with a higher gauge corresponding to a smaller needle's outer diameter. A fine needle can be defined as a gauge of ≥22, corresponding to an outer diameter of ≤0.72 mm. In some instances, a larger needle (e.g., 21-gauge or 18-gauge) can be used. CNB can be performed with a larger-gauge needle, ranging from 14-gauge to 20-gauge (e.g., outer diameter of 2.1 mm to 0.91 mm).

The needle gauge and thickness of the needle wall can dictate the maximal diameter of any tissue fragments obtained by FNA or CNB. Appreciable microscopic tissue architecture can be obtained even with high-gauge needles.

For any patient suspected of prostate cancer for example, a physician can perform transrectal ultrasound-guided prostate biopsy using a special prostate biopsy “gun” to drive ultra-fine biopsy needles (e.g., about half an inch long and a sixteenth of an inch in diameter) through the wall of the rectum and into the prostate. Each hollow needle can remove a fine cylindrical “core” of prostate tissue in about a second. About 12 to 20 biopsy cores can be taken from defined regions of the prostate. Targeted biopsies can be carried out under MRI.

Segmental resection (or segmentectomy) can be a surgical procedure to remove part of an organ or gland. It may also be used to remove a tumor and the surrounding normal tissue. In lung cancer surgery, segmental resection can refer to removing a section of a lobe of the lung. The resection margin can be the edge of the removed tissue. It can be important that this shows free of cancerous cells upon examination by a pathologist. As a result, such tissue may contain tumors as well as normal tissues.

When intact removal is not performed for a variety of reasons, a wedge of tissue may be taken in an incisional biopsy. In some cases, a sample can be collected by devices that can “bite” a sample. A variety of sizes of needles can collect tissue in the lumen (e.g., core biopsy).

Pathologic examination of a biopsy (e.g., as part of histological characterization) can determine whether a lesion is benign or malignant and can also differentiate between different types of cancers. The pathological examination can be a technique to determine the exact nature of the tumor (e.g., subclassification of tumor and histologic “grading”) and the extent of its spread (e.g., pathologic “staging”).

After the biopsy is performed, the sample of tissue that was removed from the patient can be sent to the pathology laboratory. A pathologist can diagnose diseases (e.g., cancer) by examining the tissue under a microscope. When the pathology laboratory receives the biopsy sample, the tissue can be processed and a thin slice of tissue can be removed from the sample and attached to a glass slide. The slide with the tissue attached can be treated with dyes that stain the tissue, which allows the cells and tissue structure to be observed more clearly. The pathologist can then examine the tissue under a microscope, looking for abnormal tissue structures and cellular signatures. The pathologist can prepare a report that lists any abnormal or important findings from the biopsy. A cancer diagnosis can be based on the pathological evaluation of biopsies.

To achieve tissue identification, an accurate correlation between the MRI properties and pathology parameters can be needed for each individual patient. The pathology parameters can include tissue type, tumor grade, genetic signature, cell shape, cell size, necrosis, benign tissue, and inflammation. The methods of the present disclosure can obtain such correlation for an individual patient by performing MRI measurement and pathology analysis on the same biopsy samples.

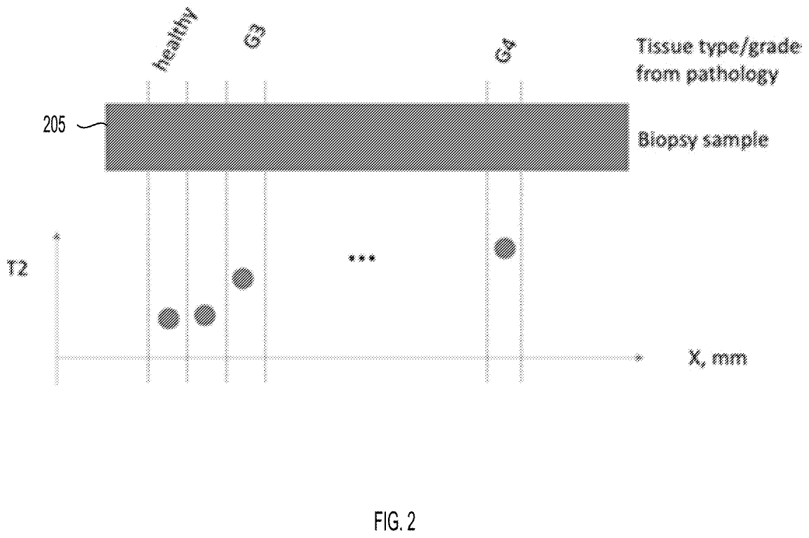

FIG. 2 illustrates the colocation of tissue type information from pathology and MRI parameters obtained from biopsy MRI. Axis X is along the length of the biopsy core sample. A biopsy sample 205 can be used for both pathology analysis and MR measurement. The pathology result (e.g., tissue type, tumor grade, etc.) can be recorded at different positions (e.g., different x-positions) along the length of the biopsy sample 205. The grade can include undifferentiated (G4), poorly differentiated (G3), moderately differentiated (G2), well differentiated (G1). The same biopsy sample 205 can be used to perform MR measurements to obtain MR parameters along the length of the biopsy sample 205. An example of T2 data is illustrated, but the same idea can be applied to other MR parameters (e.g., T1, D).

A first portion of the biopsy sample 205 can be labeled with a first MRI characterization (e.g., label). A second portion of the biopsy sample 205 can be labeled with a second MRI characterization. A third histological characterization can be determined for a portion of an MRI image of the patient based on the biopsy sample 205.

This combined data (e.g., pathology data and MRI data) can be used to obtain the MRI parameters (e.g., T1, T2, D) for a specific tissue type determined by pathology. This information can be readily used to improve tissue identification by MRI (e.g., without having to perform additional biopsy or pathology analysis).

The method can include the performance of MR measurement of the biopsy sample using a small MR system. This measurement step can be termed “biopsy MRI”. While clinical MRI imaging can use large and expensive scanners, the parameters T1, T2, and D of tissue samples can be readily measured by smaller MR instruments and by research laboratories. These instruments can be used to accurately measure T1, T2, and D, and their distribution and correlation functions, of small samples. In addition, the biopsy sample can be directly measured in such an MR system if using a biopsy needle that is compatible with MR measurement (e.g., can be present during MR measurement, and/or may not interfere with the MR measurement). Such needles may be made of plastics, or other non-metallic materials. Part or all of the needle may not be covered by metal.

The method can include a biopsy-assisted clinical MRI analysis workflow for personalized MRI. The method can include performing clinical MRI for patients who are suspected of having cancer (or diseased/anomalous tissue) diagnosis to obtain the image from the patient to locate the disease/affected area. The method can include performing a biopsy to extract the disease/reference tissue from the patient. The method can include measuring MRI parameters (e.g., T1, T2, D) using a small MRI system (e.g., smaller than a full-body scanner, tabletop MRI machine). For example, the method can include making biopsy MRI measurements to obtain the biopsy-MRI data at multiple positions (e.g., X_MRI) on the biopsy samples. The biopsy MRI can be performed at the same magnetic field (e.g., magnetic field value) as the clinical MRI. The method can include performing a pathology analysis on the biopsy samples. The method can include recording pathology parameters (e.g., tissue type information, tissue grade information) at the multiple positions (defined to be X_path) of the biopsy sample, which may be referred to as histological characterization of the biopsy sample. There can be a significant overlap of X_MRI and X_path. The method can include obtaining MRI properties and pathology parameters along every millimeter (or other unit) of the biopsy sample. From each overlapping position of the biopsy sample, the method can include listing the corresponding MRI property and pathology parameter to form a table of correlation. Combining MRI properties and the pathological data on the same biopsy sample can determine the specific MRI parameters for each tissue type. This information can be termed “personalized MRI calibration data.” The method can include applying the specific MRI parameters for each tissue type to analyze the patient MRI image to identify different tissue types on the image.

Clinical MRI can be performed at an early stage of the diagnosis and thus can be performed before biopsy MRI. Further MRI scans may be prescribed later for different methods (e.g., pulse sequences), resolutions, or contrast enhancement. MRI can also be used as a follow-up to monitor the treatment progress. Thus, clinical MRI scans may not be limited to before biopsy. This method (e.g., workflow) can be applied to MRI scans after biopsy.

Biopsy samples may contain the disease tissue. Biopsy samples may contain healthy (e.g., normal) tissues. The healthy tissues can be located a distance from the disease site. Biopsy samples can contain disease tissues with different grades. The biopsy can be performed with different needle sizes (e.g., 16 gauge, OD 1.6 mm). The biopsy can be performed with different needle types. The biopsy samples (e.g., cores) can be in a range of 10 to 20 mm long. The diameter of the cores can be about 1 mm. The samples used in this method can include biopsy samples, tissue samples from partial resection, or tissue samples from total resection.

The biopsy MRI can be aimed at obtaining MRI parameters of the tissue in the in vivo condition at the body temperature. The biopsy MRI can be performed at body temperature (e.g., the patient's body temperature). The biopsy MRI can be performed as soon as the biopsy samples are obtained from the patient. A significant loss of water from the tissue can be avoided. The biopsy MRI system may be maintained at the body temperature prior to the sample insertion to accelerate the measurements.

Biopsy needles can be made of metal. The biopsy samples can be transferred to a non-metallic holder before insertion into the biopsy MRI system for measurement. If a non-metallic or MRI-compatible needle is used, then the needle may be placed in the biopsy MRI system directly for measurement without sample transfer.

The biopsy MRI measurement can obtain the parameters (T1, T2, D, etc.) at different positions of the biopsy samples. For example, the biopsy MRI measurement can obtain the parameters along the length of the cores. The spatial resolution can be about 1 mm. For example, the spatial resolution of the biopsy MRI measurement can be about the same as that of clinical MRI. Clinical MRI resolutions can be in a range of 1 mm to 4 mm. The biopsy MRI measurement can have a resolution that is greater than that of clinical MRI. The spatial resolution can be obtained by employing multiple RF coils. The spatial resolution can be obtained through the use of a pulsed field gradient, such as frequency encoding and phase encoding methods.

Once both pathology analysis and biopsy MRI have been performed on the same core, then co-locating the two results can allow for the determination of MRI parameters for the specific tissue type as shown in FIG. 2. This information (e.g., personalized MRI calibration data) can be used for the analysis of clinical MRI images.

The personalized MRI calibration data may include the mean values of the MRI parameters of each tissue type, and also their variance. These MRI parameters (e.g., mean values and variance) can be used to identify the likelihood of certain tissue types for each voxel in the clinical MRI images.

The biopsy-MRI-assisted clinical MRI analysis can be visualized by overlaying the tissue type information on the clinical MRI image, for example, using different colors. It can also be shown using contours outlining the regions of different tissues. Such information can be displayed on a separate computer display panel and/or window for selected voxels.

The method can include determining MRI parameters for a known tissue type. The MRI measurement of a biopsy sample can allow for the determination of MRI parameters (e.g., MRI characterization) for a known tissue type.

The method can include determining MRI parameters using MRI-guided biopsy. For example, the method can include performing clinical MRI on a patient to obtain a first image. The method can include performing the biopsy under the guidance of MRI to obtain a second MRI image while the biopsy needle is extracting the tissue from the patient. The method can include aligning the first image and the second image to locate the position of the biopsy sample and the corresponding MRI data of the biopsy sample. The method can include performing the pathology analysis on the biopsy samples. The method can include recording pathology parameters (e.g., tissue type information, tissue grade information) at the multiple positions of the biopsy sample. There can be a significant overlap of X_MRI and X_path. The method can include obtaining MRI properties and pathology parameters along every millimeter of the biopsy sample. From each overlapping position of the biopsy sample, the method can include listing the corresponding MRI property and pathology parameter to form the table of correlation. Combining MRI properties and the pathological data on the same biopsy sample can determine the specific MRI parameters for each tissue type. The method can include applying the specific MRI parameters for each tissue type to analyze the patient MRI image/results to identify different tissue types on the image.

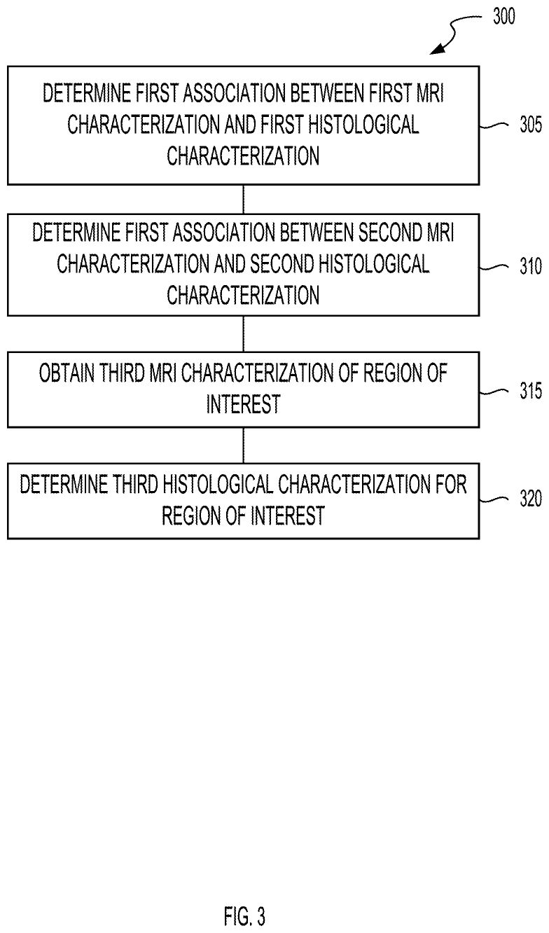

FIG. 3 illustrates a method 300 for MRI calibration. The method 300 can calibrate MRI images and/or scans for personalized diagnostics. In brief summary, the method 300 can include determining a first association between a first MRI characterization and a first histological (e.g., pathological) characterization (BLOCK 305). The method 300 can include determining a second association between a second MRI characterization and a second histological characterization (BLOCK 310). The method 300 can include obtaining a third MRI characterization of a region of interest (BLOCK 315). The method 300 can include determining a third histological characterization for the region of interest (BLOCK 320).

The method 300 can include determining a first association (e.g., mapping, correlation, etc.) between a first MRI characterization and a first histological characterization (BLOCK 305). The first MRI characterization can be obtained from a first reference sample of a patient. The first reference sample can include healthy tissue (e.g., non-cancerous tissue, benign tissue) or unhealthy tissue (e.g., cancerous tissue, anomalous tissue). The first MRI characterization can include one or more values corresponding to one or more MRI parameters. The MRI parameters can include a spin-lattice relaxation (T1), a spin-spin relaxation (T2), or a diffusion constant (D). The MRI parameters can include a distribution of T1, T2, or D. The MRI parameters can include correlation functions of T1, T2, or D. The first MRI characterization can relate to one or more MRI parameters corresponding to at least one of the spin-lattice relaxation, the spin-spin relaxation, or the diffusion constant. The first histological characterization can be obtained from the first reference sample of the patient. The first histological characterization can include at least one of tissue type, tissue grade, or genomic information. The first histological characterization can be determined by a pathologist. The first reference sample can have a width in a range of 0.1 mm to 2 mm. For example, the first reference sample can have a width in a range of 0.1 mm to 0.5 mm, 0.1 mm to 1 mm, 0.1 mm to 1.5 mm, 0.1 mm to 2 mm, 0.5 mm to 1 mm, 0.5 mm to 1.5 mm, 0.5 mm to 2 mm, 1 mm to 1.5 mm, 1 mm to 2 mm, or 1.5 mm to 2 mm. The first reference sample can include at least one of a biopsy sample, a sample from a partial resection, or a sample from a total resection. The first reference sample can be tagged with the first MRI characterization and the first histological characterization.

The method 300 can include determining a second association between a second MRI characterization and a second histological characterization (BLOCK 310). The second MRI characterization can be obtained from a second reference sample of the patient. The second reference sample can include healthy tissue or unhealthy tissue. The second reference sample of the patient can be the same as or different from the first reference sample of the patient. The second MRI characterization can include one or more values corresponding to one or more MRI parameters. The MRI parameters can include the spin-lattice relaxation, the spin-spin relaxation, or the diffusion constant. The MRI parameters can include a distribution of T1, T2, or D. The MRI parameters can include correlation functions of T1, T2, or D. The second MRI characterization can relate to one or more MRI parameters corresponding to at least one of the spin-lattice relaxation, the spin-spin relaxation, or the diffusion constant. The second histological characterization can be obtained from the second reference sample of the patient. The second histological characterization can include at least one of tissue type, tissue grade, or genomic information. The second histological characterization can be determined by the pathologist. The second reference sample can have a width in a range of 0.1 mm to 2 mm. For example, the second reference sample can have a width in a range of 0.1 mm to 0.5 mm, 0.1 mm to 1 mm, 0.1 mm to 1.5 mm, 0.1 mm to 2 mm, 0.5 mm to 1 mm, 0.5 mm to 1.5 mm, 0.5 mm to 2 mm, 1 mm to 1.5 mm, 1 mm to 2 mm, or 1.5 mm to 2 mm. The second reference sample can include at least one of a biopsy sample, a sample from a partial resection, or a sample from a total resection. The second reference sample can be tagged with the second MRI characterization and the second histological characterization.

In some embodiments, the method 300 can include determining one or more associations between one or more MRI characterizations and one or more histological characterizations of a sample. The samples can include a reference sample of the patient. The one or more associations can include the first association and the second association. The one or more MRI characterizations can include the first MRI characterization and the second characterization. The one or more histological characterizations can include the first histological characterization and the second histological characterization. Tissue sample can be tagged (e.g., labeled, associated) with the one or more MRI characterizations and the one or more histological characterizations. This can allow for the determination of the histological characterization from the MRI characterization, e.g., via association with each common tissue sample.

The method 300 can include obtaining a third MRI characterization of a region of interest (ROI) (BLOCK 315). For example, the method 300 can include obtaining the third MRI characterization of the ROI from an MRI scan of the patient. The method 300 can include obtaining the third MRI characterization of the ROI from an MRI image of the patient. The region of interest can include a voxel. The region of interest can include a portion of an organ. The region of interest can include tissue. The region of interest can have a width in a range of 0.1 mm to 2 mm. For example, the region of interest can have a width in a range of 0.1 mm to 0.5 mm, 0.1 mm to 1 mm, 0.1 mm to 1.5 mm, 0.1 mm to 2 mm, 0.5 mm to 1 mm, 0.5 mm to 1.5 mm, 0.5 mm to 2 mm, 1 mm to 1.5 mm, 1 mm to 2 mm, or 1.5 mm to 2 mm. The region of interest can have a length in a range of 0.1 mm to 2 mm. For example, the region of interest can have a length in a range of 0.1 mm to 0.5 mm, 0.1 mm to 1 mm, 0.1 mm to 1.5 mm, 0.1 mm to 2 mm, 0.5 mm to 1 mm, 0.5 mm to 1.5 mm, 0.5 mm to 2 mm, 1 mm to 1.5 mm, 1 mm to 2 mm, or 1.5 mm to 2 mm.

The method 300 can include determining a third histological characterization for the region of interest (BLOCK 320). For example, the method 300 can include determining the third histological characterization for the ROI using the third MRI characterization and at least one of the first association or the second association. The second histological characterization can be determined by the pathologist. This can allow for the determination of the third histological characterization of the region of interest without the need for additional and/or unnecessary biopsy samples.

The method 300 can include determining one or more values for one or more MRI parameters corresponding to each tissue type of a plurality of reference samples of the patient. The plurality of references samples can include the first reference sample and the second reference sample. The tissue type can include, for example, healthy tissue or tumor grade (e.g., undifferentiated, poorly differentiated, moderately differentiated, well differentiated).

The method 300 can include extracting one or more reference samples from the patient. For example, the method 300 can include extracting the first reference sample from the patient. The method 300 can include extracting the second reference sample from the patient. The one or more reference samples can be part of a single tissue core or different tissue cores. The method 300 can include extracting the one or more reference samples using a needle (e.g., biopsy needle).

The method 300 can include performing an MRI scan of the one or more reference samples. For example, the method 300 can include performing a first MRI scan of the first reference sample. The method 300 can include performing a second MRI scan of the second reference sample. The method 300 can include obtaining an MRI image/scan/characterization of the one or more reference samples. For example, the method 300 can include obtaining a first MRI image/scan of the first reference sample. The method 300 can include obtaining a second MRI image/scan of the second reference sample.

The method 300 can include obtaining an MRI characterization from the MRI scan. For example, the method 300 can include obtaining the first MRI characterization from the first MRI scan. The first MRI scan be an MRI scan of the patient (e.g., rather than of a biopsy sample). The first MRI scan be an MRI scan of the first reference sample. The method 300 can include obtaining the second MRI characterization from the second MRI scan. The second MRI scan be an MRI scan of the patient. The second MRI scan be an MRI scan of the second reference sample. The method 300 can include obtaining an MRI characterization from the MRI image. For example, the method 300 can include obtaining the first MRI characterization from the first MRI image. The method 300 can include obtaining the second MRI characterization from the second MRI image.

The method 300 can include performing a histological analysis on the one or more reference samples to obtain one or more histological characterizations. For example, the method 300 can include performing a first histological analysis on the first reference sample to obtain the first histological characterization. The method 300 can include performing a second histological analysis on the second reference sample to obtain the second histological characterization. The histological analysis can include a characterization of tissues obtained from sectioning, staining, and examining these tissues under a microscope or using other pathological process/analysis. The method 300 can include performing a pathological analysis on the one or more reference samples to obtain the one or more histological characterizations. For example, the method 300 can include performing a first pathological analysis on the first reference sample to obtain the first histological characterization. The method 300 can include performing a second pathological analysis on the second reference sample to obtain the second histological characterization. The pathological analysis can include an analysis of the tissue performed by one or more pathologists.

The method 300 can include performing an MRI scan of a region corresponding to the reference sample. For example, the method 300 can include performing a first MRI scan of a first region corresponding to the first reference sample. The MRI scan can be performed prior to collection of the first reference sample using an MR compatible needle (e.g., MRI-compatible needle). The MRI scan can be performed during collection of the first reference sample using the MR compatible needle. The MRI scan can be performed after collection of the first reference sample using the MR compatible needle. The method 300 can include performing a second MRI scan of a second region corresponding to the second reference sample. The MRI scan can be performed prior to collection of the second reference sample using the MR compatible needle. The MRI scan can be performed during collection of the second reference sample using the MR compatible needle. The MRI scan can be performed after collection of the second reference sample using the MR compatible needle.

The method 300 can include performing the MRI scan of the one or more reference samples at body (e.g., patient's body) temperature. For example, the method 300 can include performing the MRI scan of the one or more reference samples at a temperature in a range of 97° F. to 99° F. The one or more reference samples can have a temperature in a range of 97° F. to 99° F. For example, the temperature can be in a range of 97° F. to 97.5° F., 97° F. to 98° F., 97° F. to 98.5° F., 97° F. to 99° F., 97.5° F. to 98.5° F., 97.5° F. to 98.5° F., 97.5° F. to 99° F., 98° F. to 98.5° F., 98° F. to 98.5° F., 98° F. to 99° F., or 98.5° F. to 99° F. The method 300 can include performing the first MRI scan of the first reference sample at a temperature in a range of 97° F. to 99° F. The method 300 can include performing the first MRI scan of the second reference sample at a temperature in a range of 97° F. to 99° F.

The method 300 can include determining a plurality of MRI characterizations along a length of a sample. The plurality of MRI characterizations can include the first MRI characterization and the second MRI characterization. The method 300 can include determining the first MRI characterization along the length of the first reference sample. The method 300 can include determining the second MRI characterization along the length of the second reference sample. The method 300 can include determining the plurality of MRI characterizations along the length of the core (e.g., biopsy core). The core can have a length in the range of 10 mm to 20 mm. The core can be made of one or more reference samples.

The method 300 can include determining a plurality of histological characterizations along the length (or other dimension) of the sample. The plurality of histological characterizations can include the first histological characterization and the second histological characterization. The method 300 can include determining the first histological characterization along the length of the first reference sample. The method 300 can include determining the second histological characterization along the length of the second reference sample. The method 300 can include determining the plurality of histological characterizations along the length of the core.

The method 300 can include determining a probability of one or more tissue types for each voxel (or other unit of location or region) obtained from the MRI scan of the patient. For example, the method 300 can include determining that a first voxel of the MRI scan of the patient has a first probability of being a first tissue type and a second probability of being a second tissue type. The method 300 can include determining that a second voxel of the MRI scan of the patient has a third probability of being the first tissue type and a fourth probability of being the second tissue type.

The method 300 can include determining one or more values of one or more MRI parameters corresponding to a known tissue type. For example, the method 300 can include determining a first value of a first MRI parameter corresponding to a first tissue type. The method 300 can include determining a second value of the first MRI parameter corresponding to a second tissue type. The first value of the first MRI parameter can be different from the second value of the first MRI parameter. The first tissue type can be different from the second tissue type.

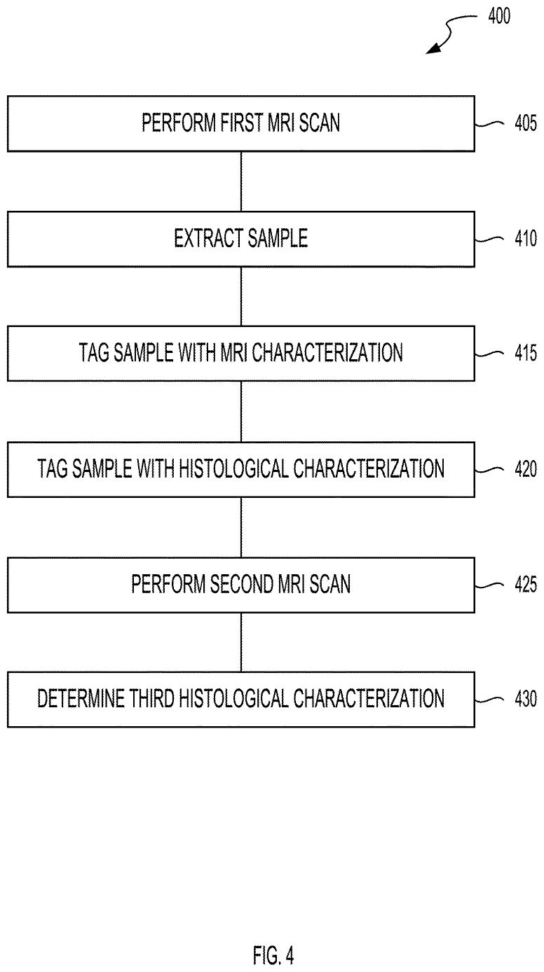

FIG. 4 illustrates a method 400 for MRI calibration. The method 400 can calibrate MRI images and/or scans for personalized diagnostics. In brief summary, the method 400 can include performing a first MRI scan (BLOCK 405). The method 400 can include extracting a sample (BLOCK 410). The method 400 can include tagging a sample with an MRI characterization (BLOCK 415). The method 400 can include tagging (e.g., associating or labeling) the sample with a histological characterization (BLOCK 420). The method 400 can include performing a second MRI scan (BLOCK 425). The method 400 can include determining a third histological characterization (BLOCK 430).

The method 400 can include performing a first MRI scan (BLOCK 405). The method 400 can include performing the first MRI scan of the patient. For example, the method 400 can include performing the first MRI scan of the patient while a needle (e.g., biopsy needle) is inserted into the patient. The needle can include an MR compatible needle. For example, the need can be made of plastic or non-metallic materials. The first MRI scan can be performed to obtain a first MRI image.

The method 400 can include extracting a sample (BLOCK 410). The method 400 can include extracting the sample from the patient. For example, the method 400 can include extracting the sample from the patient using the biopsy needle. The sample can be extracted before, during, or after the first MRI scan.

The method 400 can include tagging (e.g., labeling, marking) a sample with an MRI characterization (BLOCK 415). For example, the method 400 can include tagging a first portion of the sample with a first MRI characterization. The first MRI characterization can be obtained from the first MRI scan. The first MRI characterization can include one or more values corresponding to one or more MRI parameters. The first MRI characterization can relate to one or more MRI parameters corresponding to at least one of a spin-lattice relaxation, a spin-spin relaxation, or a diffusion constant. The method 400 can include tagging a second portion of the sample with a second MRI characterization. The second MRI characterization can be obtained from the first MRI scan. The second MRI characterization can include one or more values corresponding to one or more MRI parameters. The second MRI characterization can relate to one or more MRI parameters corresponding to at least one of the spin-lattice relaxation, the spin-spin relaxation, or the diffusion constant.

The method 400 can include tagging the sample with a histological characterization (BLOCK 420). For example, the method 400 can include tagging the first portion of the sample with a first histological characterization, e.g., to relate/associate the first histological characterization with the first MRI characterization. The first histological characterization can include at least one of tissue type, tissue grade, or genomic information. The method 400 can include tagging the second portion of the sample with a second histological characterization, e.g., to relate/associate the second histological characterization with the second MRI characterization. The second histological characterization can include at least one of tissue type, tissue grade, or genomic information. The first portion of the sample can be adjacent to the second portion of the sample. The first portion of the sample can be located a distance from the second portion of the sample.

The method 400 can include performing a second MRI scan (BLOCK 425). For example, the method 400 can include performing the second MRI scan of the patient to obtain a third MRI characterization of a region of interest (ROI). The second MRI scan can be performed before the first MRI scan, for example. The second MRI scan can be performed to obtain a second MRI image. The second image and the first image can be aligned to locate the position of the sample and the corresponding MRI data of the sample. For example, the first image can be superimposed on the second image such that features of the first image and the second image are overlapping. The MRI parameters of the second scan can be mapped to the location of first portion of the sample and the second portion of the sample.

The method 400 can include determining a third histological characterization (BLOCK 430). For example, the method 400 can include determining the third histological characterization for the ROI using at least one of: the first MRI characterization, the second MRI characterization, the third MRI characterization, the first histological characterization, or the second histological characterization.

The method 400 can include determining one or more MRI parameters corresponding to each tissue type of the sample. The sample can include a first sample and a second sample. The first sample can be a first tissue type and have one or more MRI parameters corresponding to the first tissue type. The second sample can be a second tissue type and have one or more MRI parameters corresponding to the second tissue type.

The methods (e.g., method 300, 400) can be used to expand the application of MRI, which has been used to help to locate and target areas most likely to contain tumors during the biopsy procedure and after cancer treatment for disease progression follow-up. For example, the methods can have applications for the detection of prostate cancer, breast cancer, and brain cancer.

For prostate cancer, MRI can be the protocol for prostate cancer diagnostics and disease management. Systematic biopsy alone can underdiagnose about 40% of the cancers and MRI-targeted biopsy alone can underdiagnose about 30% of the cancers, while combined biopsy can underdiagnose 14% of the cancers. Since needle biopsy and MRI can be routinely prescribed for prostate cancer diagnostics and management, the methods of the present disclosure can be readily applied to improve the cancer diagnosis, thereby reducing the risk of both overdiagnosis/underdiagnosis and undertreatment of cancer.

For breast cancer, breast MRI can be a useful tool for confirming the presence of a lesion and its localization when mammography and tomosynthesis findings are not adequate for evaluation, or the lesion is viewed from a single position and cannot be localized, or the lesion is subtle and suspicious and cannot be detected by ultrasonography. Improving the diagnosis to detect the progression of breast cancer early and accurately can contribute significantly to the treatment of breast cancer patients. One of the problems in the diagnosis of breast cancer can include how to distinguish breast cancer from some benign lesions such as breast fibroma, fibroadenosis, and lobular hyperplasia. Thus, the methods of the present disclosure can improve the accuracy of tissue identification and breast cancer diagnosis.

For brain cancer, MRI can be the cornerstone for evaluating patients with brain masses such as primary and metastatic tumors. Challenges in effectively detecting and diagnosing brain tumors and metastases and in accurately characterizing their subsequent response to treatment can exist. These difficulties can include discriminating metastases from potential mimics such as primary brain tumors and infection, detecting small metastases, and differentiating treatment response from tumor recurrence and progression. The methods of the present disclosure can improve diagnosis and be used as a routine assessment or continued reassessment of brain metastasis. For example, breast cancer brain metastasis (BCBM) can represent a major clinical challenge.

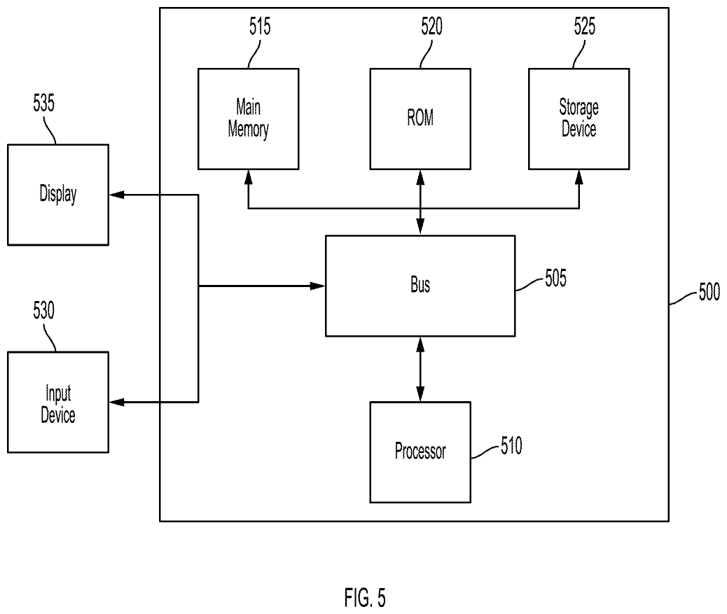

FIG. 5 depicts an example block diagram of an example computing system 500 (e.g., computing device). The computing system 500 can include or be used to implement a data processing system or its components. The computing system 500 includes at least one bus 505 or other communication component for communicating information and at least one processor 510 or processing circuit coupled to the bus 505 for processing information. The computing system 500 can also include one or more processors 510 or processing circuits coupled to the bus for processing information. The computing system 500 also includes at least one main memory 515, such as a random-access memory (RAM) or other dynamic storage device, coupled to the bus 505 for storing information, and instructions to be executed by the processor 510. The main memory 515 can be used for storing information during the execution of instructions by the processor 510. The computing system 500 may further include at least one read-only memory (ROM) 520 or other static storage device coupled to the bus 505 for storing static information and instructions for the processor 510. A storage device 525, such as a solid-state device, magnetic disk or optical disk, can be coupled to the bus 505 to persistently store information and instructions.

The computing system 500 may be coupled via the bus 505 to a display 535, such as a liquid crystal display, or active-matrix display, for displaying information to a user. An input device 530, such as a keyboard or voice interface may be coupled to the bus 505 for communicating information and commands to the processor 510. The input device 530 can include a touch screen display 535. The input device 530 can also include a cursor control, such as a mouse, a trackball, or cursor direction keys, for communicating direction information and command selections to the processor 510 and for controlling cursor movement on the display 535.

The processes, systems and methods described herein can be implemented by the computing system 500 in response to the processor 510 executing an arrangement of instructions contained in main memory 515. Such instructions can be read into main memory 515 from another computer-readable medium, such as the storage device 525. Execution of the arrangement of instructions contained in main memory 515 causes the computing system 500 to perform the illustrative processes described herein. One or more processors in a multi-processing arrangement may also be employed to execute the instructions contained in main memory 515. Hard-wired circuitry can be used in place of or in combination with software instructions together with the systems and methods described herein. Systems and methods described herein are not limited to any specific combination of hardware circuitry and software. The computing system 500 can have sensors that feed data. The computing system 500 can have the ability for data to be sent or passed to connected units and sensors. The computing system 500 can pull data from connected units and sensors.

Although an example computing system has been described in FIG. 5, the subject matter including the operations described in this specification can be implemented in other types of digital electronic circuitry, or in computer software, firmware, or hardware, including the structures disclosed in this specification and their structural equivalents, or in combinations of one or more of them.

Embodiments of the subject matter and the operations described in this specification can be implemented in digital electronic circuitry, or in computer software, firmware, or hardware, including the structures disclosed in this specification and their structural equivalents, or in combinations of one or more of them. The subject matter described in this specification can be implemented as one or more computer programs, e.g., one or more circuits of computer program instructions, encoded on one or more computer storage media for execution by, or to control the operation of, data processing apparatus. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium may not be a propagated signal, a computer storage medium can be a source or destination of computer program instructions encoded in an artificially generated propagated signal. The computer storage medium can also be, or be included in, one or more separate components or media (e.g., multiple CDs, disks, or other storage devices).

The operations described in this specification can be performed by a data processing apparatus on data stored on one or more computer-readable storage devices or received from other sources. The term “data processing apparatus” or “computing device” encompasses various apparatuses, devices, and machines for processing data, including by way of example a programmable processor, a computer, a system on a chip, or multiple ones, or combinations of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a circuit, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more circuits, subprograms, or portions of code). A computer program can be deployed to be executed on one computer or on multiple computers that are located at one site or distributed across multiple sites and interconnected by a communication network.

Processors suitable for the execution of a computer program include, by way of example, microprocessors, and any one or more processors of a digital computer. A processor can receive instructions and data from a read-only memory or a random-access memory or both. The elements of a computer are a processor for performing actions in accordance with instructions and one or more memory devices for storing instructions and data. A computer can include, or be operatively coupled to receive data from or transfer data to, or both, one or more mass storage devices for storing data, e.g., magnetic, magneto optical disks, or optical disks. A computer need not have such devices. Moreover, a computer can be embedded in another device, e.g., a personal digital assistant (PDA), a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The implementations described herein can be implemented in any of numerous ways including, for example, using hardware, software or a combination thereof. When implemented in software, the software code can be executed on any suitable processor or collection of processors, whether provided in a single computer or distributed among multiple computers.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

A computer employed to implement at least a portion of the functionality described herein may comprise a memory, one or more processing units (also referred to herein simply as “processors”), one or more communication interfaces, one or more display units, and one or more user input devices. The memory may comprise any computer-readable media, and may store computer instructions (also referred to herein as “processor-executable instructions”) for implementing the various functionalities described herein. The processing unit(s) may be used to execute the instructions. The communication interface(s) may be coupled to a wired or wireless network, bus, or other communication means and may therefore allow the computer to transmit communications to or receive communications from other devices. The display unit(s) may be provided, for example, to allow a user to view various information in connection with execution of the instructions. The user input device(s) may be provided, for example, to allow the user to make manual adjustments, make selections, enter data or various other information, or interact in any of a variety of manners with the processor during execution of the instructions.

The various methods or processes outlined herein may be coded as software that is executable on one or more processors that employ any one of a variety of operating systems or platforms. Additionally, such software may be written using any of a number of suitable programming languages or programming or scripting tools, and also may be compiled as executable machine language code or intermediate code that is executed on a framework or virtual machine.

In this respect, various inventive concepts may be embodied as a computer-readable storage medium (or multiple computer-readable storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the solution discussed above. The computer-readable medium or media can be transportable, such that the program or programs stored thereon can be loaded onto one or more different computers or other processors to implement various aspects of the present solution as discussed above.

The terms “program” or “software” are used herein to refer to any type of computer code or set of computer-executable instructions that can be employed to program a computer or other processor to implement various aspects of embodiments as discussed above. One or more computer programs that when executed perform methods of the present solution need not reside on a single computer or processor, but may be distributed in a modular fashion amongst a number of different computers or processors to implement various aspects of the present solution.

Computer-executable instructions may be in many forms, such as program modules, executed by one or more computers or other devices. Program modules can include routines, programs, objects, components, data structures, or other components that perform particular tasks or implement particular abstract data types. The functionality of the program modules can be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

While operations are depicted in the drawings in a particular order, such operations are not required to be performed in the particular order shown or in sequential order, and all illustrated operations are not required to be performed. Actions described herein can be performed in a different order.

Having now described some illustrative implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements and features discussed in connection with one implementation are not intended to be excluded from a similar role in other implementations or implementations.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular can include implementations including a plurality of these elements, and any references in plural to any implementation or element or act herein can include implementations including only a single element. References in the singular or plural form are not intended to limit the presently disclosed systems or methods, their components, acts, or elements to single or plural configurations. References to any act or element being based on any information, act or element may include implementations where the act or element is based at least in part on any information, act, or element.

Any implementation disclosed herein may be combined with any other implementation, and references to “an implementation,” “some implementations,” “an alternate implementation,” “various implementations,” “one implementation” or the like are not necessarily mutually exclusive and are intended to indicate that a particular feature, structure, or characteristic described in connection with the implementation may be included in at least one implementation. Such terms as used herein are not necessarily all referring to the same implementation. Any implementation may be combined with any other implementation, inclusively or exclusively, in any manner consistent with the aspects and implementations disclosed herein.

References to “or” may be construed as inclusive so that any terms described using “or” may indicate any of a single, more than one, and all of the described terms. References to at least one of a conjunctive list of terms may be construed as an inclusive OR to indicate any of a single, more than one, and all of the described terms. For example, a reference to “at least one of ‘A’ and ‘B’” can include only ‘A’, only ‘B’, as well as both ‘A’ and ‘B’. Elements other than ‘A’ and ‘B’ can also be included.

The systems and methods described herein may be embodied in other specific forms without departing from the characteristics thereof. The foregoing implementations are illustrative rather than limiting of the described systems and methods.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

The systems and methods described herein may be embodied in other specific forms without departing from the characteristics thereof. The foregoing implementations are illustrative rather than limiting of the described systems and methods. Scope of the systems and methods described herein is thus indicated by the appended claims, rather than the foregoing description, and changes that come within the meaning and range of equivalency of the claims are embraced therein.

1. A method, comprising:
determining a first association between a first magnetic resonance imaging (MRI) characterization and a first histological characterization obtained from a first reference sample of a patient;
determining a second association between a second MRI characterization and a second histological characterization obtained from a second reference sample of the patient;
obtaining a third MRI characterization of a region of interest (ROI) from an MRI scan of the patient; and
determining a third histological characterization for the ROI using the third MRI characterization and at least one of the first association or the second association.

2. The method of claim 1, wherein the first MRI characterization comprises one or more values corresponding to one or more MRI parameters.

3. The method of claim 1, wherein the first histological characterization comprises at least one of tissue type, tissue grade, or genomic information.

4. The method of claim 1, comprising:
determining values for one or more MRI parameters corresponding to each tissue type of a plurality of reference samples of the patient.

5. The method of claim 1, comprising:
extracting the first reference sample from the patient.

6. The method of claim 1, comprising at least one of:
performing an MRI scan of the first reference sample;
obtaining the first MRI characterization from the MRI scan; or
performing a histological or pathological analysis of the first reference sample to obtain the first histological characterization.

7. The method of claim 1, comprising:
performing an MRI scan of a region corresponding to the first reference sample, prior to or during or after collection of the first reference sample using an MR compatible needle.

8. The method of claim 1, comprising:
performing an MRI scan of the first reference sample at a temperature in a range of 97° F. to 99° F.

9. The method of claim 1, wherein at least one of the ROI, the first reference sample or the second reference sample, has a width in a range of 0.1 mm to 2 mm.

10. The method of claim 1, wherein the first MRI characterization relates to one or more MRI parameters corresponding to at least one of a spin-lattice relaxation (T1), a spin-spin relaxation (T2), or a diffusion constant (D).

11. The method of claim 1, comprising:
determining a plurality of MRI characterizations comprising the first MRI characterization and the second MRI characterization along a length of a sample; and
determining a plurality of histological characterizations comprising the first histological characterization and the second histological characterization along the length of the sample.

12. The method of claim 1, wherein the first reference sample is at least one of a biopsy sample, a sample from a partial resection, or a sample from a total resection.

13. The method of claim 1, comprising:
determining a probability of one or more tissue types for each voxel obtained from the MRI scan of the patient.

14. The method of claim 1, comprising:
determining one or more values of one or more MRI parameters corresponding to a known tissue type.

15. A method, comprising:
performing a first MRI scan of a patient while a biopsy needle is inserted into the patient;
extracting a sample from the patient using the biopsy needle;
tagging a first portion of the sample with a first MRI characterization obtained from the first MRI scan;
tagging a second portion of the sample with a second MRI characterization obtained from the first MRI scan;
tagging the first portion of the sample with a first histological characterization;
tagging the second portion of the sample with a second histological characterization;
performing a second MRI scan of the patient to obtain a third MRI characterization of a region of interest (ROI); and
determining a third histological characterization for the ROI using at least one of: the first MRI characterization, the second MRI characterization, the third MRI characterization, the first histological characterization, or the second histological characterization.

16. The method of claim 15, wherein the first MRI characterization comprises one or more values corresponding to one or more MRI parameters.

17. The method of claim 15, wherein the first histological characterization comprises at least one of tissue type, tissue grade, or genomic information.

18. The method of claim 15, comprising:
determining one or more MRI parameters corresponding to each tissue type of the sample.

19. The method of claim 15, wherein the biopsy needle is an MR compatible needle.

20. The method of claim 15, wherein the first MRI characterization relates to one or more MRI parameters corresponding to at least one of a spin-lattice relaxation (T1), a spin-spin relaxation (T2), or a diffusion constant (D).
